(19) United States
(12) Patent Application Publication
(10) Pub. No.: US 2006/0264899 A1
(43) Pub. Date: Nov. 23, 2006

(54) Transcutaneous fluid drain kit

(75) Inventors: David S. Paul, Downey, CA (US); Ridwan Hardy, Temple City, CA (US); Evan Walker, Rancho Dominguez, CA (US); J. Rae Walker, Rancho Dominguez, CA (US); Ning Xu, Diamond Bar, CA (US)

Correspondence Address: OSTROLENK, FABER, GERB & SOFFEN, LLP, 1180 Avenue of the Americas, New York, NY, 10036-8403, US

(73) Assignee: Axiom Medical, Inc.

(21) Appl. No.: 11/434,454

(22) Filed: May 15, 2006

Related U.S. Application Data
(63) Continuation of application No. 10/476,472, filed on Feb. 6, 2004, now abandoned.

(51) Int. Cl. A61M 31/00; A61M 3/00; A61M 25/00

(52) U.S. Cl. 604/506; 604/43; 604/284

(57) ABSTRACT

A transuctaneous fluid kit for controllably evacuating fluid from a fluid site to a fluid collector, while preventing an outrush of fluid from the fluid site. A cannula is provided for fluid collection at the site to be drained. A Y-connector is provided at the proximal end of the needle. A one-way valve is mounted to the first leg of the Y-counter to prevent the outrush of fluid upon insertion of the cannula to the fluid site. A second leg of the Y-connector is provided for carrying evacuated fluid to a collection device via tubing.

### CROSS-REFERENCE TO RELATED APPLICATION

This application is based on and claims priority to provisional application number 60/288,199 filed May 2, 2001, incorporated by reference.

### BACKGROUND OF THE INVENTION

The present invention relates to a drainage system for body fluids, particularly for the collection of postoperative seromas, cysts and other confined regions.

Drainage systems of background interest are disclosed in U.S. Pat. Nos. 4,246,899; 4,341,212; and 4,692,155. A one-way valve is disclosed in U.S. Pat. No. 5,000,745. The full disclosure of each prior art reference mentioned herein is expressly incorporated by reference.

A problem in conventional systems, particularly the '212 patent, is that when the cannula is inserted into the fluid to be drained, there is an outrush of fluid, which can lead to unsanitary conditions and in the worst case can lead to a risk of infection for medical personnel.

Further, these systems lack flexibility. It would be desirable to be able not merely to drain the fluid, but also to wash out the site of the fluid to be drained by injecting saline or another fluid to the site in conjunction with the drainage operation.

### SUMMARY OF THE INVENTION

The present invention provides a solution to these problems, by providing a drainage system comprising a plurality of lumens and a one-way valve.

Drainage is achieved without immobilizing the patient, repeated surgery or multiple needle aspirations. The fluid site is accessed by a tubular structure which serves as an intake passage from the patient to the collection chamber. The collection chamber may provide a suction source to encourage flow of fluids from the fluid site to the collection chamber via the intermediate tubing.

The tubular structure comprises a cannula which is introduced to the site by use of a piercing component which has a cutting shape at its distal end (the direction of advance). The piercing component may be a trocar or a needle or any other suitable cutting device. The cannula remains at the site and the piercing component is removed.

After the piercing component is removed, a second needle or cannula can be inserted for selectively injecting saline or another fluid or substance.

A Y-connector is connected to the proximal end of the cannula The Y-connector is connected to the collection device by tubing.

The tubular structure, particularly the cannula, comprises two lumens, one for evacuation from the site and a second for introducing a substance such as a fluid for use in cleaning or treating the site. At its proximal end the tubular element is expanded into two separate and distinct channels by the Y-connector.

A one-way valve is provided in a first leg of the Y-connector for preventing fluid from rushing out of the Y-connector when the piercing component or the second cannula is inserted or removed.

The Y-connector and the one-way valve may advantageously be combined into a more compact connector providing the functions of both.

Another valve may be provided between the collecting device and the cannula. The valve may be a one-way valve for preventing fluid which has been collected from reentering the tubular structure from the collection device; or may be an on-off valve for selectively preventing or regulating fluid flow in either direction. The collection device is connected to the tubular structure by a second piercing element that penetrates a sealed plug on the collection chamber.

As used herein, the term “fluid” includes any flowable material (a liquid or even a gas) that may be encountered in the medical context, including clear liquids as well as liquids containing tissues and/or solid matter.

Other features and advantages of the present invention will become apparent from the following description of embodiments of the invention which refers to the accompanying drawings.

### BRIEF DESCRIPTION OF THE DRAWINGS

FIGS. 1 and 2 are an exploded view and an assembled view showing some of the components of an embodiment of the invention.

FIGS. 3 and 4 are an exploded view and an assembled view showing additional components of the embodiment.

FIG. 5 is an exploded view similar to FIG. 1, showing a second embodiment of the invention

### DETAILED DESCRIPTION OF EMBODIMENTS OF THE INVENTION

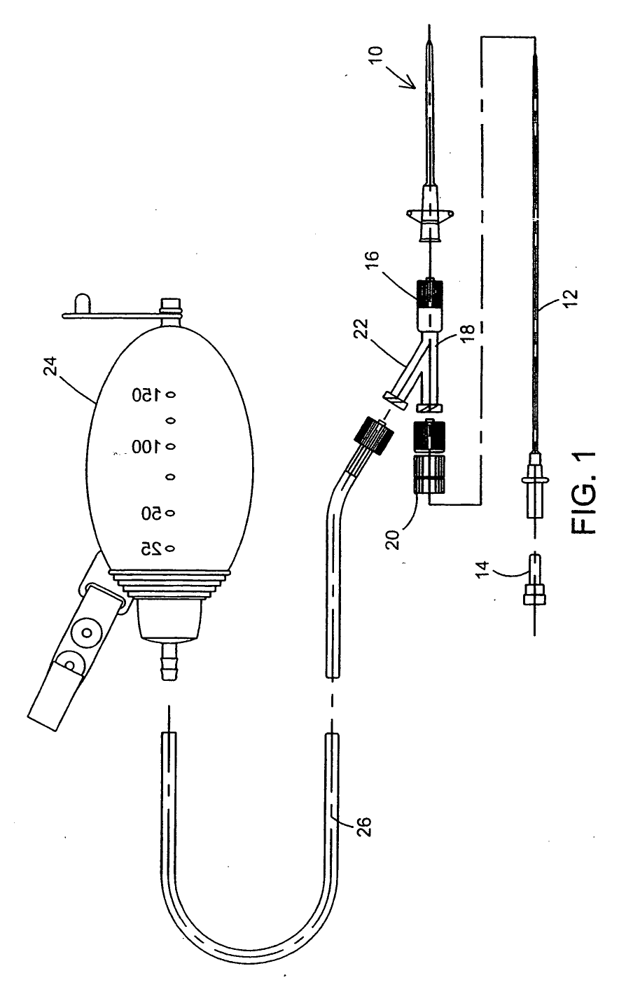

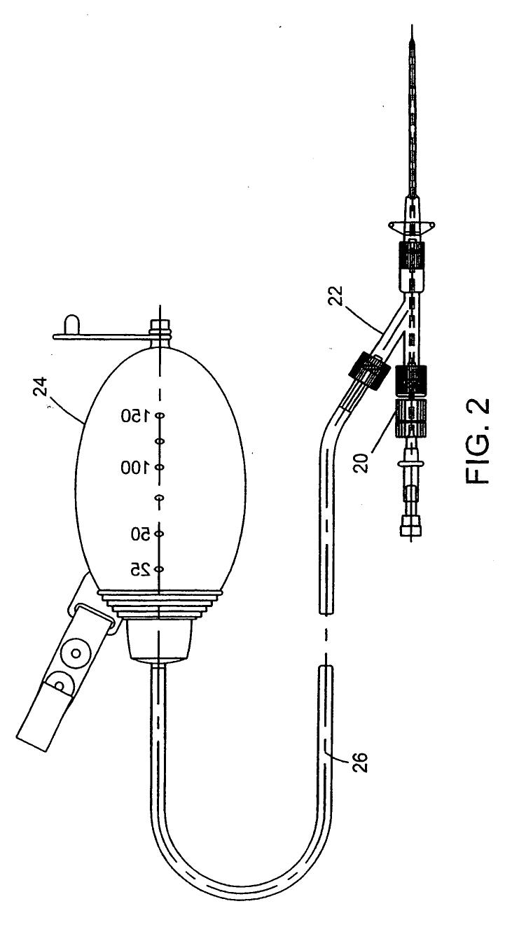

FIGS. 1 and 2 are an exploded view and an assembled view showing some of the components of an embodiment of the invention.

A cannula 10 is provided for fluid collection at the site to be drained. In order to insert the cannula 10, a piercing element, which is a needle 12 in this embodiment, is inserted through the cannula 10. A plug 14 is provided at the proximal end of the needle 12. The plug 14 is preferably porous in order to facilitate equilibrium between ambient pressure and internal needle pressure. The porous plug may further help the user identify the location of insertion, if fluid appears at the end of the needle.

A Y-connector 16 is provided at the proximal end of the cannula 10. The needle 12 is inserted through a first leg 18 of the Y-connector 16. A one-way valve 20 is mounted to the first leg 18. The needle 12 is inserted through the valve 20. The valve 20 prevents outrush of the fluid upon insertion of the needle and cannula to the site.

A second leg 22 of the Y-connector 16 is provided for carrying evacuated fluid to a collection device 24, preferably of a vacuum type, via tubing 26.

Another valve (not shown) may be provided at the tubing 26. The other valve may be either a one-way valve or an on-off valve. It may be operable to control fluid flow in the tubing, and/or to block the second leg 22 when the tubing 26 is not connected, and/or to prevent collected fluid from returning from the collection device.

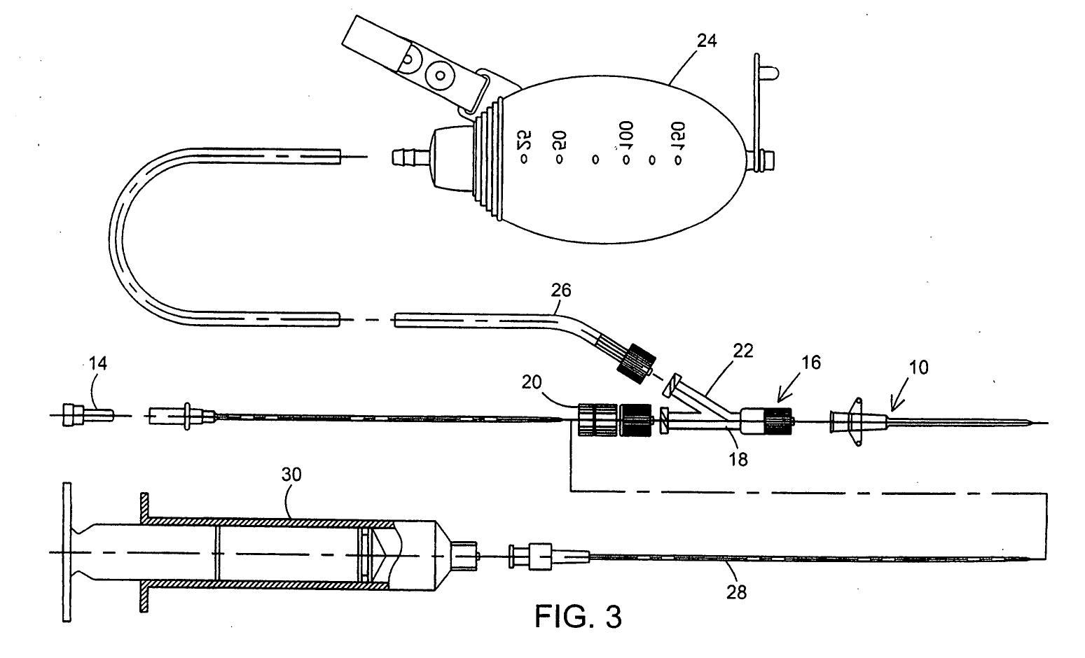

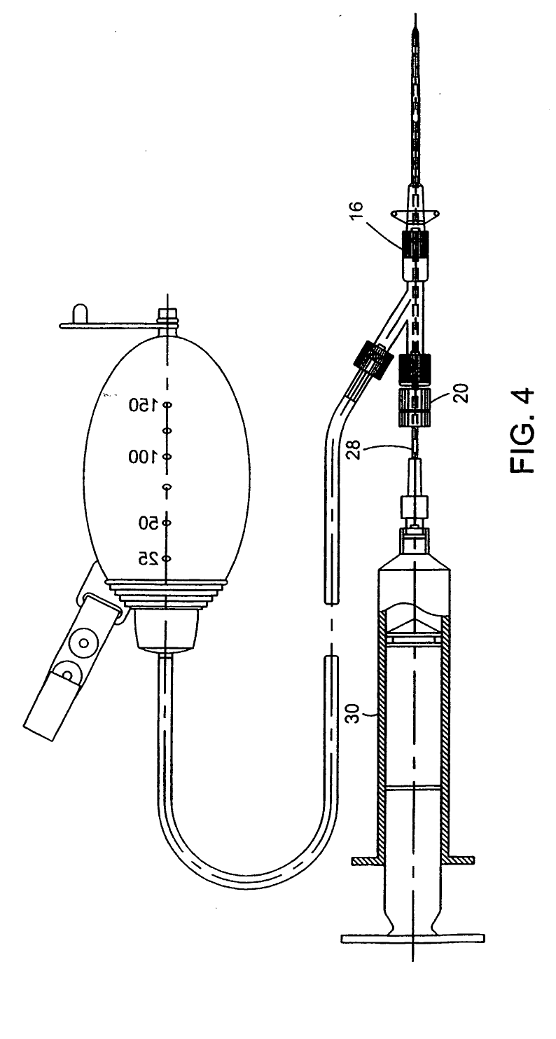

After the cannula 10 is in place at the drainage site, the needle 12 can be removed and a second cannula 28 can be inserted, to be used for example for injection of saline or another fluid. See FIGS. 3 and 4, which are an exploded view and an assembled view showing additional components of the embodiment. When the needle 12 is removed, the one-way valve 20 prevents fluid from rushing out through the first leg 18. Then the second cannula 28 can be inserted to the site. A syringe 30, for example, is connected to the second cannula 28 for injecting the fluid.

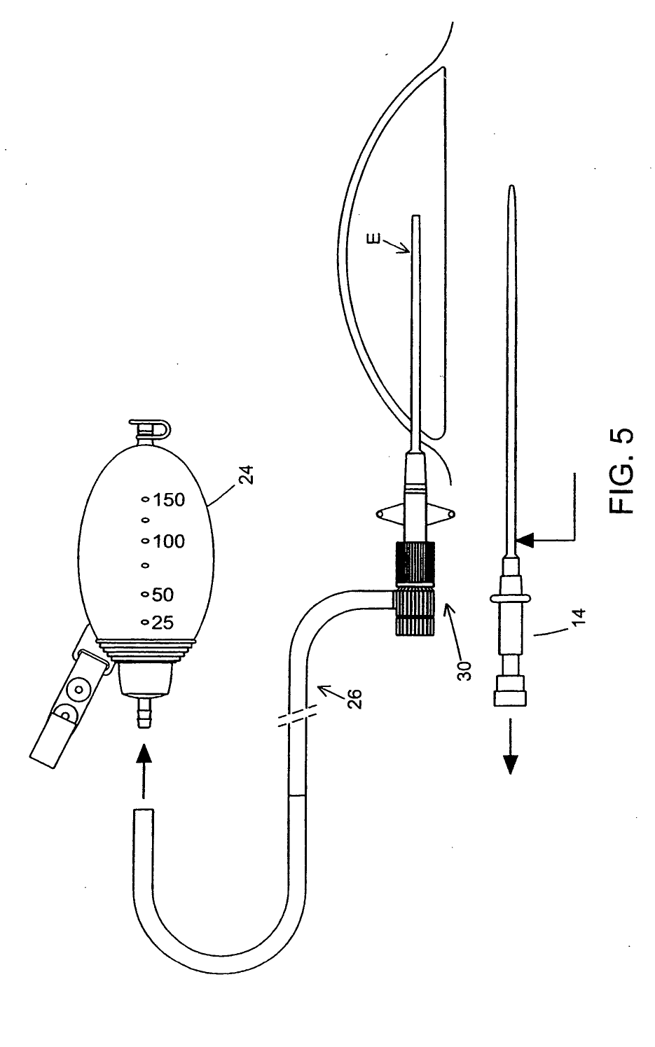

A second embodiment of the invention is shown in FIG. 5. The second embodiment is made smaller than the first embodiment by replacing the Y-connector 16 and the one-way valve 20 of the first embodiment with a more compact connector 30. The connector 30 provides the same functionality as the one-way valve 20 and the Y-connector 16 of the first embodiment in a more compact package. A suitable connector is disclosed in U.S. Pat. No. 5,000,745 the disclosures of which are incorporated by reference.

Although the present invention has been described in relation to particular embodiments thereof, many other variations and modifications and other uses will become apparent to those skilled in the art. Therefore, the present invention is not limited by the specific disclosure herein.

1-14. (canceled)

15. A fluid kit for transcutaneous treatment of a fluid site comprising:
at least one cannula removably insertable into the fluid site for draining the fluid therefrom; and
a fluid flow controller comprising a one-way valve in fluid communication with the at least one cannula and operative for preventing an outrush of the fluid from the fluid site during insertion of the at least one cannula into the fluid site.

16. The fluid kit defined in claim 15, wherein the fluid flow controller is comprised in a connector mounted on the proximal end of the at least one cannula.

17. The fluid kit defined in claim 15, wherein the at least one cannula is connected to spaced apart first and second lumens for selectively evacuating the fluid from the fluid site and for delivering a substance for cleaning or treating the fluid site.

18. The fluid kit defined in claim 17, wherein the fluid flow controller includes a Y-connector having said first and second lumens and mounted on a proximal end of the at least one cannula at a distance from the fluid site.

19. The fluid defined in claim 18, wherein said one-way valve is operative to block said first lumen of said Y-connector so that when the at least one cannula is being inserted into the fluid site, the fluid flows through the second lumen of the Y-connector.

20. The fluid kit defined in claim 18, further comprising a collection device for receiving fluid flow from the fluid site to the collection device through the second lumen of the Y-connector.

21. The fluid kit defined in claim 20, wherein the collection device generates suction.

22. The fluid kit defined in claim 20, further comprising tubing which is removably attached to the Y-connector and the collection device, the fluid kit further comprising a second valve provided at the tubing for preventing the fluid collected in the collection device from returning to the fluid site and for blocking the second lumen of the Y-connector if the tubing is detached.

23. The fluid kit defined in claim 22, wherein the second valve is a one-way valve or an on-off valve.

24. A fluid kit for transcutaneous treatment of a fluid site comprising:
at least one cannula removably insertable into the fluid site for draining the fluid therefrom; and
a fluid flow controller in fluid communication with the at least one cannula and operable for preventing an outrush of the fluid from the fluid site during insertion of the at least one cannula into the fluid site;
wherein the at least one cannula is connected to spaced apart first and second lumens for selectively evacuating the fluid from the fluid site and for delivering a substance for cleaning or treating the fluid site;
wherein the fluid flow controller includes a Y-connector having said first and second lumens and mounted on a proximal end of the at least one cannula at a distance from the fluid site; and
wherein the fluid flow controller further includes a first valve provided at the Y-connector and operative to block the first lumen of the Y-connector so that when the at least one cannula is being inserted into the fluid site, the fluid flows through the second lumen of the Y-connector, the first valve being a one-way valve.

25. The fluid kit defined in claim 24, further comprising a piercing element removably insertable through the first valve and the first lumen of the Y-connector for providing access of a remote end of the cannula into the fluid site, the piercing element being a trocar or a needle.

26. The fluid kit defined in claim 25, further comprising a plug mounted on a proximal end of the piercing element removed from the fluid site.

27. The fluid kit defined in claim 26, wherein the plug is comprised of porous material to facilitate equilibrium between ambient pressure and internal piercing element pressure.

28. The fluid kit defined in claim 25, further comprising a second element insertable into the at least one cannula through the first valve and first lumen of the Y-connector after the piercing element has been removed for providing a substance for cleaning or treating the fluid site.

29. A fluid kit for transcutaneous treatment of a fluid site comprising:
at least one cannula removably insertable into the fluid site for draining the fluid therefrom; and
a second element removably insertable through the cannula for communicating between a proximal end of the cannula and the fluid site; and
further comprising a plug mounted on a proximal end of the second element removed from the fluid site, the plug comprising porous material to facilitate equilibrium between ambient pressure and pressure at the fluid site.

30. A method of transcutaneously treating a fluid site within the body of a patient, comprising the steps of:
connecting, to a cannula, a fluid flow controller comprising a one-way valve in fluid communication with the cannula and operative for preventing an outrush of fluid from said cannula;
inserting said cannula to said fluid site through an incision in the body of the patient;
draining and/or irrigating said site through said cannula; and
removing said cannula from the patient's body.

31. A method of transcutaneously treating a fluid site within the body of a patient, comprising the steps of:
connecting a fluid flow controller in fluid communication with at least one cannula which is operative for preventing an outrush of the fluid from the fluid site during insertion of the at least one cannula into the fluid site;
inserting the at least one cannula into the fluid site for draining fluid therefrom; and
connecting the at least one cannula to spaced apart first and second lumens for selectively evacuating the fluid from the fluid site and for delivering a substance for cleaning or treating the fluid site;
wherein the fluid flow controller includes a Y-connector having said first and second lumens and mounted on a proximal end of the at least one cannula at a distance from the fluid site; and
providing a one-way valve at the Y-connector of the fluid flow controller which is operative to block the first lumen of the Y-connector so that when the at least one cannula is being inserted into the fluid site, the fluid flows through the second lumen of the Y connector.

32. A method of transcutaneously treating a fluid site within the body of a patient, comprising the steps of:
inserting at least one cannula into the fluid site for draining the fluid therefrom; and
inserting a second element through the cannula for communicating between a proximal end of the cannula and the fluid site; and
providing a plug mounted on a proximal end of the second element removed from the fluid site, the plug comprising porous material to facilitate equilibrium between ambient pressure and pressure at the fluid site.
